(19) United States
(12) Patent Application Publication
(10) Pub. No.: US 2020/0291342 A1
(43) Pub. Date: Sep. 17, 2020

(54) POLYMERIC CELL CULTURE SURFACE HAVING HIGH CELL ADHESION

(71) Applicant: SIO2 MEDICAL PRODUCTS, INC., Auburn, AL (US)

(72) Inventors: Ahmad Taha, Auburn, AL (US); Brian Maurer, Auburn, AL (US); Matthew Wills, Auburn, AL (US)

(21) Appl. No.: 16/756,525

(22) PCT Filed: Oct. 19, 2018
(86) PCT No.: PCT/US2018/056722

§ 371 Date: Apr. 16, 2020

Related U.S. Application Data
(60) Provisional application No. 62/575,037, filed on Oct. 20, 2017.
(60) Provisional application No. 62/718,284, filed on Aug. 13, 2018.

(51) Int. Cl. C12M 3/04; C08J 7/12; C12M 1/00

(52) U.S. Cl. CPC C12M 27/12; C08J 7/123; C08J 2325/06; C12M 47/02; C12M 23/20

(57) ABSTRACT

A polymeric substrate is contacted with a process gas and radio frequency electrical power is introduced in the process gas, forming a treated contact surface that has improved cell recovery compared to an untreated contact surface. The process gas optionally can be nitrogen gas, oxygen gas, or a gas that contains nitrogen atoms, oxygen atoms, or a combination of nitrogen and oxygen atoms. The process optionally improves cell recovery of a chicken embryo cell culture from the treated contact surface.

### BACKGROUND

The technology relates generally to a surface, or surface modification of a plastic substrate (sometimes referred to in this disclosure as a contact surface), being hydrophilic, or making the surface hydrophilic and enhancing cell adhesion to the surface. More particularly, the technology relates to a plastic substrate, e.g. a medical device or item of laboratory ware, with a treated surface used for cell culture and cell growth due to its enhanced cell adhesion. Such medical devices include, but are not limited to cell culture vessels and roller bottles.

Although some cells grow in suspension (e.g. 3D sphere culture suspension), such as hematopoietic cell lines and transformed cells, most other cells grow in favor of high surface binding (e.g. monolayer growth); that is, they require surface attachment to proliferate. Historically, glass was used as the growth surface since it has superior optical qualities, is hydrophilic and naturally charged which are favored to promote cell growth. Disposable plastic, especially polystyrene is now most commonly used for cell culture growth. Polystyrene culture vessels are of good optical quality.

However, since most plastics are hydrophobic and unsuitable for cell growth, their surfaces need to be treated or coated.

In cell growth vessels, it is desirable to enhance cell adsorption and cell binding to the plastic ware used with biological substances. Surfaces of common laboratory ware components made of polymeric plastic are hydrophobic and usually don't have good cell adhesion. It is thus a desire to provide surfaces for plastic laboratory ware and other articles that contact biological substances with higher hydrophilicity and thereby improved cell adhesion.

The present invention also relates to the technical field of fabrication of coated vessels for conducting chemical, biochemical, medical, and/or biological uses. These methods and systems are essential in a variety of applications including medical diagnostics, medical treatment, environmental monitoring, manufacturing quality control, drug discovery, and scientific research.

This invention generally relates to fabrication of cell growth and cell culture vessels and plastic lab ware. This invention also relates to producing a hydrophilic surface by plasma treatment. This invention further relates to generation of a hydrophilic surface with enhanced cell adhesion and thereby an improved cell culture and cell growth.

Traditionally glassware presents a hydrophilic surface and therefore was used, and continues to be used for cell culture and cell growth. However glassware is readily breakable, very expensive, prone to particulate problems, yields heavy metal extractables, and can cause adverse effect on cell growth and/or aggregation of proteins and other biologics.

Some of these problems can be addressed by substituting injection molded plastic ware for glassware. In particular, plastic ware is preferred in the biologics area, such as areas of medicine, medical research, drug discovery, and scientific research, due to the large number of issues with glassware. Plastic ware addresses some of the problems with glassware, but plastic ware creates certain problems as well. Plastic ware contains extractables/leachables, preventing the use of plastic ware or making it undesirable for many types of laboratory in vitro and analytical testing. Plastic ware presents a hydrophobic surface which usually gives low cell adhesion. High cell adhesion is considered to enhance cell growth. These issues limit the use of plastic ware for cell culture vessels and roller bottles.

Roller bottles are used as cell culture vessels in a wide variety of applications. Roller bottles are often made from polystyrene (PS) or polyethylene terephthalate (PET). These materials present superior optical clarity, high stability, reduced breakage and many other advantages.

The relatively large contacting surface of a roller bottle enhances cell adhesion, thereby improving cell growth. To expand the contacting surface, some roller bottles are designed with circumferential, axial, or other ribs on the body, which can multiply the growth surface.

To generate a hydrophilic surface that is beneficial for cell growth, some hydrophilic coatings, including polyethylene glycol (PEG) and zwitterion polymeric coatings are being used which provide good cell adhesion. Many of these polymeric coatings are not covalently bound to the article surface and have potential to move (dissolve, disperse) into the fluid payload, causing interference with cell growth or testing, limiting their utility. Polymeric coatings that are covalently attached to the article surface would not have the potential to move (dissolve, disperse) into the fluid payload, eliminating this source of interference with cell growth. Further, covalently bound polymeric coating would prevent movement of the polymeric surface coating, thereby preventing undesired exposure of the article surface.

There is therefore a need for hydrophilic coatings/treatments for the surface of plastic laboratory ware such as cell culture vesselsand roller bottles, that will enhance the cell adhesion to the surface of the plastic. Likewise, there is a need for covalently bound polymeric coatings/treatments for the surface of plastic laboratory ware such as cell culture vesselsand roller bottles that will prevent movement of the polymeric surface coating thereby preventing undesired particulate interference and exposure of the plastic surface.

### SUMMARY OF THE INVENTION

An aspect of the invention is a method carried out, in general, by providing a polymeric substrate including an initial contact surface, contacting the initial contact surface with a process gas, and introducing radio frequency electrical power in the process gas, forming a treated contact surface that has improved cell recovery compared to an untreated contact surface.

The polymeric substrate includes, in addition to the initial contact surface, an interior portion adjacent to the initial contact surface.

The process gas optionally can be nitrogen gas, oxygen gas, or a heterogeneous gas that contains nitrogen atoms, oxygen atoms, or a combination of nitrogen and oxygen atoms, as well as other kinds of atoms, for example noble gases. Non-limiting examples of suitable process gas include oxygen gas, nitrogen gas, nitrous oxide gas, or a combination of any two or more of these.

Optionally, the radio frequency electrical power is introduced in the process gas adjacent to the initial contact surface to generate plasma adjacent to the initial contact surface. As a result, a treated polymeric substrate is formed having a treated contact surface.

The process optionally improves cell recovery of a chicken embryo cell culture from the treated contact surface, relative to the initial contact surface, optionally resulting in cell recovery from the treated contact surface of at least 140% of the cells provided to the treated contact surface at the beginning of the cell recovery test.

### BRIEF DESCRIPTION OF DRAWING FIGURES

In the drawings,

FIG. 1 is a schematic view of plasma treatment apparatus useful for carrying out any embodiment of the invention.

FIG. 2 is a view similar to FIG. 1 showing plasma treatment apparatus for treating three vessels simultaneously.

FIG. 3 is a schematic sectional view of the apparatus of FIG. 1, showing internal details of the apparatus and an additional feature for equalizing pressure inside and outside of a vessel being treated.

FIG. 4 shows a perspective view of a CELLTREAT™ roller bottle.

FIG. 5 shows a photographic view similar to FIG. 4 of a commercial roller bottle having multiple circumferential ribs inside and outside its wall, expanding the surface area for cell attachment.

FIG. 6 shows the CELLTREAT™ roller bottle of FIG. 5 as referred to in Example 2 of this specification, identifying relevant parts of the bottle.

FIGS. 7A and 7B show two examples of aseptic caps which can be used to close the vessel of the current invention. FIG. 7A shows a Corning® aseptic transfer cap and FIG. 7B shows a Sartorius MYCAP® closure.

The following reference characters are used in the drawings:

| | |
|---|---|
| 101 | polymeric substrate |
| 102 | contact surface |
| 103 | interior portion (adjacent to the contact surface) |
| 104 | process gas |
| 105 | vessel |
| 106 | wall (of 105) |
| 107 | inner surface (of 106) |
| 108 | lumen (of 105) |
| 109 | outer surface (of 106) |
| 110 | ribs |
| 111 | gas inlet conduit |
| 112 | outlet (of 111) |
| 113 | external applicator |
| 114 | internal applicator |
| 115 | ceramic chamber |
| 116 | aluminum bottom |
| 117 | aluminum lid |
| 118 | pumping port |
| 119 | vacuum conduit |
| 120 | vacuum pump |
| 121 | Valve |
| 122 | processing area |
| 123 | gas system |
| 124 | mass flow controller |
| 125 | matching network |
| 126 | power supply |
| 127 | coaxial cable |
| 128 | vacuum bypass line |
| 129 | valve (of 128) |

Like reference characters indicate corresponding parts.

### DETAILED DESCRIPTION

The present disclosure is directed to a process for making a roller bottle or other lab ware or substrate having a contact surface that is hydrophilic and has higher cell adhesion than an untreated surface or biological coating treated surface.

Optionally, when the substrate of this invention is used for cell growth, the cells are harvested or recovered after the growth process is complete. The recovery rate optionally is higher than for a biological coating treated, otherwise identical substrate. The recovery rate optionally is higher than for a Corning Cellbind substrate.

Optionally, if the substrate is embodied as a vessel, the vessel further comprises a closure. The closure can be of any kind. For example, the closure can be any stopper, cap, lid, top, cork or any combination of them. For example, a plastic or elastomer stopper can be inserted into a cap to form a closure.

Cell growth requires an aseptic environment. Frequent opening and closing the cap of the cell culture/growth vessel is one of the sources of contamination. Optionally cell culture/growth vessels (e.g. roller bottles) can be closed with an aseptic transfer cap to prevent the contamination due to opening and closing the cap during media feeding, inoculation, sample addition/collection, transferring, etc. Optionally, the closure is suitable for an aseptic process, optionally at high temperature, low temperature, autoclaving, irradiation or any other unusual conditions. For example, the closure can be an aseptic transfer cap with other accessories to eliminate the need to open the cap during the cell culture/growth process. Optionally, the closure can be a Corning® aseptic transfer cap. Optionally, the closure can be a Sartorius MYCAP® closure. The MYCAP® closure comprises a silicone elastomer dispensed into a cap. The cap is assembled by inserting a tubing and a gas exchange cartridge into preformed holes located on the cap.

Optionally, the method comprises the steps of (a) providing a substrate, for example a vessel, having a contact surface; (b) drawing a vacuum adjacent to the contact surface; (c) providing a gas comprising O2, optionally containing nitrogen, in the vicinity of the contact surface; and (d) generating a plasma from the gas, thus forming a treated contact surface. The formed contact surface is a high cell binding surface.

Optionally, if the substrate is a roller bottle or other vessel, in step (c), the gas is optionally introduced into the vessel through a gas inlet inserted into the vessel (as illustrated in Fig. XX. Optionally in this embodiment, RF is used to generate the plasma.

Surprisingly, it was found that RF power combined with use of a gas inlet introducing the gas mixture into a vessel affords great advantages in enhancing the results in cell growth experiments. The results are better than uncoated otherwise identical surfaces and also better than a Corning Cellbind treated surface. Not limited by the theory, when using RF power to treat a vessel without a gas inlet inserted into the vessel to deliver the gas mixture, less reactive functional groups may be generated on the surface, thus a less desired treatment may be obtained. Using a gas inlet inserted into the vessel to deliver the gas mixture helps generate more reactive functional groups on the surface, thus improving surface activation and surface uniformity to achieve better cell adhesion/cell growth results.

There are several advantages for using a RF power source versus a microwave source: Since RF operates at a lower power, there is less heating of the substrate/vessel. Because the focus of the present invention is a plasma surface treatment of plastic substrates, lower processing temperatures are desired to prevent melting/distortion of the substrate. The higher frequency microwave can also cause off-gassing of volatile substances like residual water, oligomers and other materials in the plastic substrate. This off-gassing can interfere with the treatment.

The term “contact surface” indicates a surface that is in a position to come in contact with a sample or other material, and has surface properties determining its interaction with the sample or other material with which it comes into contact. Some examples of contact surfaces are part or all of an interior surface of a vessel (for example, bounding a vessel lumen) or an exterior surface of a vessel, sheet, block, or other object. Optionally, the contact surface is made of the same material as the interior portion before the contact surface is treated with plasma.

The term “interior portion” indicates a portion of a bulk article or coating that is not a contact surface, but instead forms part of the interior of the bulk article or coating. In embodiments in which a contact surface of a substrate is treated to modify its properties, the interior portion of the substrate includes any portion that is not modified by the treatment.

“Plasma,” as referenced in any embodiment, has its conventional meaning in physics of one of the four fundamental states of matter, characterized by extensive ionization of its constituent particles, a generally gaseous form, and incandescence (i.e. it produces a glow discharge, meaning that it emits light).

A treated contact surface is defined for all embodiments as a contact surface that has been plasma treated as described in this specification, and that exhibits enhanced cell growth as a result of such treatment.

The term “vessel” as used throughout this specification may be any type of article that is adapted to contain or convey a liquid, a gas, a solid, or any two or more of these. One example of a vessel is an article with at least one opening (e.g., one, two or more, depending on the application) and a wall including an interior contact surface.

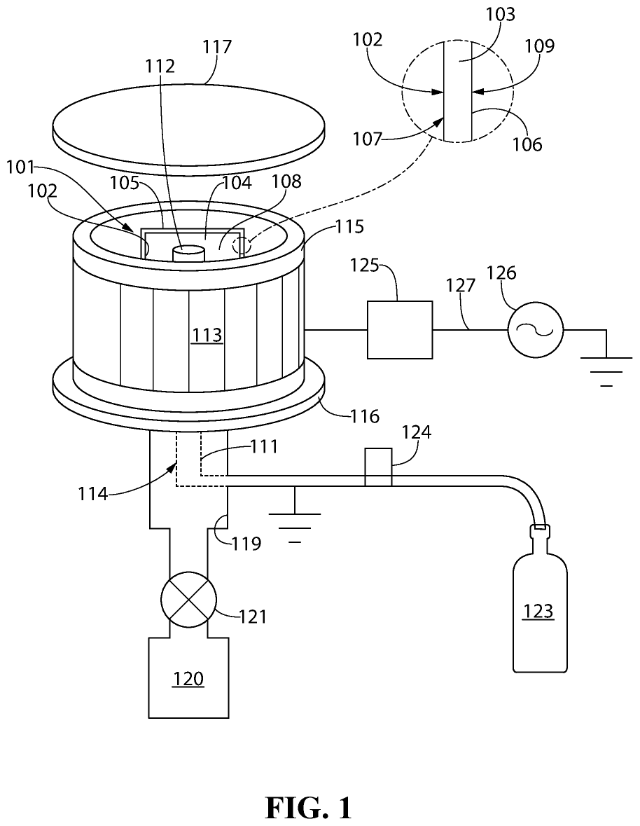

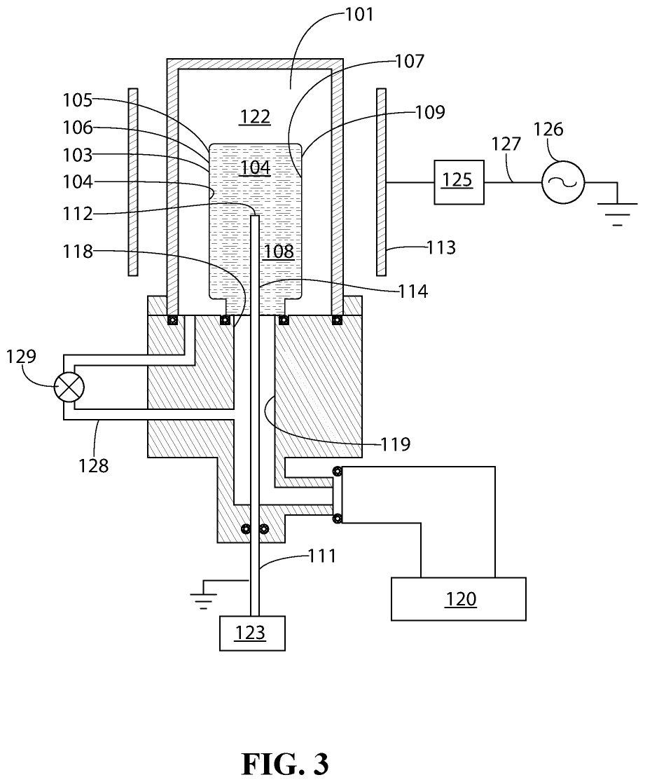

Referring to FIGS. 1-3, the present method can be carried out, in general, by providing a polymeric substrate 101 including an initial contact surface 102, contacting the initial contact surface 102 with a process gas 104 (shown as the gas source in FIG. 1, and as the gas in a vessel in FIGS. 1 and 3), and introducing radio frequency electrical power in the process gas 104, forming a treated contact surface 102 that has improved cell recovery compared to an untreated contact surface 102.

Optionally in any embodiment, the polymeric substrate 101 includes, in addition to the initial contact surface 102, an interior portion 103 adjacent to the initial contact surface 102.

Optionally in any embodiment, the process gas 104 can be nitrogen gas, oxygen gas, or a heterogeneous gas that contains nitrogen atoms, oxygen atoms, or a combination of nitrogen and oxygen atoms, as well as other kinds of atoms. Non-limiting examples of suitable process gases 104 include oxygen gas, nitrogen gas, nitrous oxide gas, or a combination of any two or more of these. Optionally, the process gas 104 can include a carrier gas, for example a noble gas, for example helium, neon, argon, krypton, or xenon or a mixture of any two or more of these.

Optionally in any embodiment, the radio frequency electrical power is introduced in the process gas 104 adjacent to the initial contact surface 102 to generate plasma adjacent to the initial contact surface 102. As a result, a treated polymeric substrate 101 is formed having a treated contact surface 102.

Optionally in any embodiment, the x-ray photoelectron spectroscopy XPS atomic composition of the treated contact surface 102 is:

from 10% to 25% oxygen, from 0 to 5% nitrogen, and from 70% to 90% carbon;

optionally from 15% to 24% oxygen, from 0.1% to 5% nitrogen, and from 70% to 80% carbon;

optionally from 20% to 24% oxygen, from 0.1% to 1% nitrogen, and from 70% to 79% carbon.

Optionally in any embodiment, the XPS atomic composition of the interior portion 103 of the treated polymeric substrate 101 comprises less oxygen and more carbon than the treated contact surface 102.

Optionally in any embodiment, the XPS atomic composition of the interior portion 103 of the treated polymeric substrate 101 at a depth of 0.6 nm comprises from 1% to 10% oxygen.

Optionally in any embodiment, the XPS atomic composition of the interior portion 103 of the treated polymeric substrate 101 at a depth of 1.2 nm comprises from 0.5% to 5% oxygen.

Optionally in any embodiment, the XPS atomic composition of the interior portion 103 of the treated polymeric substrate 101 at a depth of 1.7 nm comprises from 0.3% to 3% oxygen.

Optionally in any embodiment, the XPS atomic composition of the interior portion 103 of the treated polymeric substrate 101 at a depth of 2.3 nm comprises from 0.1% to 1% oxygen.

Optionally in any embodiment, the XPS atomic composition of the interior portion 103 of the treated polymeric substrate 101 at a depth of 2.9 nm comprises from 0.1% to 1% oxygen.

Optionally in any embodiment, the viability of a chicken embryo cell culture grown in contact with the treated contact surface 102 and harvested, relative to the initial contact surface 102, is at least 88%, optionally from 88% to 99%, optionally from 88% to 97%, optionally from 94% to 96%.

Optionally in any embodiment, the recovery of a chicken embryo cell culture grown in contact with the treated contact surface 102 and harvested, relative to the initial contact surface 102, is at least 132%, optionally from 132% to 300%, optionally from 140% to 250%, optionally from 140% to 230%.

Optionally in any embodiment, the surface contact angle of water with the treated contact surface 102 is from 38° to 62°, optionally from 50° to 70°, optionally from 55° to 65°, optionally from 60° to 64°, optionally from 30° to 50°, optionally from 30 to 40°, optionally from 35° to 45°, optionally from 37° to 41°.

Optionally in any embodiment, the treated polymeric substrate 101 comprises a vessel 105 having a wall 106 having an inner surface 107 enclosing a lumen 108, an outer surface 109, and an interior portion 103 between and spaced from the inner surface 107 and the outer surface 109. Unless otherwise indicated in this specification, locations within the interior portion 103 are identified by their distance from the inner surface 107.

The inner surface 107 optionally is generally cylindrical, and optionally the treated contact surface 102 comprises at least a portion of the inner surface 107 of the vessel 105.

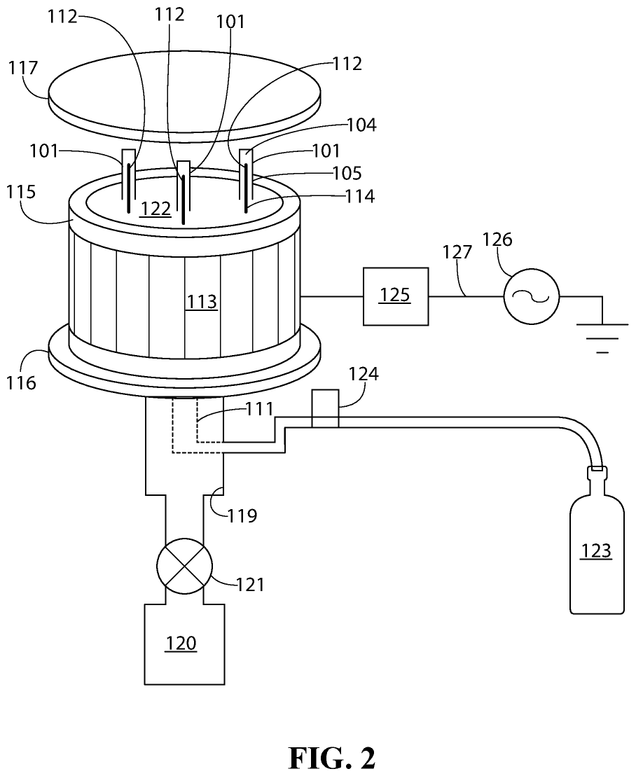

Optionally in any embodiment, the vessel 105 comprises a roller bottle as illustrated in FIGS. 1, 2, and others. Optionally, the roller bottle comprises an inner surface 107 defining the treated contact surface 102, the contact surface 102 having multiple ribs 110. Ribs or other structural complexity in part or all of the contact surface 102, for example in the cell-contacting side or end walls of the roller bottle or other vessel 105, have been found useful for increasing the surface area of the contact surface 102. Optionally in any embodiment, the vessel 105 has a volumetric capacity from 1 mL to 100 L, optionally from 100 mL to 5 L, optionally about 1 L, optionally about 2 L. Optionally in any embodiment, the treated polymeric substrate 101 can comprise a plate, a dish, a flask, a bottle as in FIGS. 1 and 3, a tube as in FIG. 2, or any other type of lab ware or production equipment.

Optionally in any embodiment, the treated polymeric substrate 101 comprises injection moldable thermoplastic or thermosetting material, for example a thermoplastic material, for example a thermoplastic resin, for example an injection-molded thermoplastic resin. Optionally in any embodiment, the thermoplastic material comprises a hydrocarbon polymer, for example an olefin polymer, polypropylene (PP), polyethylene (PE), cyclic olefin copolymer (COC), cyclic olefin polymer (COP), polymethylpentene, polystyrene, hydrogenated polystyrene, polycyclohexylethylene (PCHE), or combinations of two or more of these, or a heteroatom-substituted hydrocarbon polymer, for example a polyester, polyethylene terephthalate (PET), polyethylene naphthalate, polybutylene terephthalate (PBT, polyvinylidene chloride (PVdC), polyvinyl chloride (PVC), polycarbonate, polylactic acid, epoxy resin, nylon, polyurethane polyacrylonitrile, polyacrylonitrile (PAN), an ionomeric resin, or any combination, composite, blend, or laminate of any two or more of the above materials. Optionally in any embodiment, the thermoplastic resin comprises polystyrene, which is commonly used for many lab ware applications, including roller bottles, microplates, petri dishes, and others.

Optionally in any embodiment, the process gas 104 comprises oxygen atoms, nitrogen atoms, or both oxygen and nitrogen atoms, and preferably comprises oxygen, nitrogen, nitrous oxide, or a combination of any two or more of these. Optionally in any embodiment, the process gas 104 is essentially free of water.

Optionally in any embodiment, the present method is carried out by contacting a contact surface 102 with a process gas 104. This can be done, for example, by conveying the process gas 104 through a gas inlet conduit 111 having an outlet 112 adjacent to the initial contact surface 102.

Optionally in any embodiment, the frequency of the RF electrical power used for generating plasma is from 1 to 50 MHz, optionally 13.56 MHz. Optionally in any embodiment, the radio frequency electrical power used to excite the plasma is from 1 to 1000 Watts, optionally from 100 to 900 Watts, optionally from 50 to 600 Watts, optionally 200 to 700 Watts, optionally 400 to 600 Watts, optionally 100 to 500 Watts, optionally from 500 to 700 Watts, optionally from 1 to 100 Watts, optionally from 1 to 30 Watts, optionally from 1 to 10 Watts, optionally from 1 to 5 Watts.

Optionally in any embodiment, the radio frequency electrical power is introduced at least in part by an external applicator 113 generally surrounding the initial contact surface 102. Optionally in any embodiment, the radio frequency electrical power is introduced at least in part by an internal applicator 114 located at least partially within the lumen 108. Optionally in any embodiment, the internal applicator 114 located at least partially within the lumen 108 further comprises a gas inlet conduit 111 for contacting the initial contact surface 102 with the process gas 104.

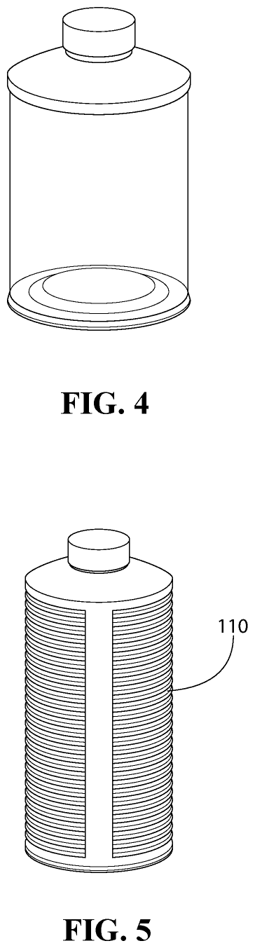

Optionally in any embodiment, apparatus as illustrated in FIG. 1 can be used to treat the initial contact surface 102 of a vessel 105. FIGS. 1 and 3 show an example of the vessel 105, configured as a roller bottle. A better view of a typical 1-liter or 2-liter capacity roller bottle is shown in FIGS. 4-6.

References in this specification to the capacity of a roller bottle or other vessel do not necessarily indicate the amount of fluid required to fill it completely full. The designated capacity of such vessels commonly allows for a headspace when the vessel is filled to its capacity. In a roller bottle, for example, the bottle is laid on its side and rolled by a mechanism when cells are being grown in the vessel so cells adhered to the contact surface 102 alternately pass through the headspace and the liquid content of the bottle, such as a growth medium, facilitating growth.

The roller bottle or other vessel 105 has a wall 106 having an inner surface 107, enclosing a lumen 108, and an outer surface 109. The vessel wall 106 has an interior portion 103 between and spaced from the inner surface 107 and the outer surface 109. At least a portion, and optionally all, of the inner surface 107 defines a contact surface 102, which is either referred to as an initial contact surface before the present treatment or a treated contact surface after the present treatment. The contact surface 102 is any part of the inner surface 107 treated according to the present disclosure.

The apparatus shown in FIG. 1, 2, or 3 is suitable for treating the vessel 105 according to any embodiment, although other apparatus can be used. This apparatus can include a cylindrical ceramic chamber 115 shown in FIGS. 1 and 2, with an aluminum bottom 116 and an aluminum lid 117 (which is closed during use, but shown open in Fig. x, as it can be when loading or unloading). The chamber 115 can be approximately 12 inches (30 cm) in diameter and 8 inches (20 cm) deep, although any other suitable dimensions can instead be used.

The pumping port 118 of the chamber 115 feeding the vacuum conduit 119 to the vacuum pump 120, optionally controlled by a valve 121, can be at the aluminum bottom 116 and can be approximately 4 inches (10 cm) in diameter, with the ½-inch (12 mm) diameter gas inlet conduit 111 concentrically protruding through the pumping port 118 into the processing area 122. A plasma screen (not shown) can be installed in over the pumping port 118 and can be constructed from copper screen and steel wool. Process gas 104 can be fed to the gas inlet conduit 111 via a gas system 123 under the chamber 115. Mass flow controllers such as 124 can be used for the compressed process gas 104.

The ceramic chamber 115 can have a copper external applicator 113 that can be concentrically wrapped around the outside of the chamber 115 and can be approximately 7 inches (18 cm) tall. The external applicator 113 can be connected to a COMDEL® matching network 125 that can allow the 50-ohm output of the COMDEL® 1000-watt RF (13.56 MHz) power supply 126 to be matched for optimal power coupling (low reflected power). COMDEL® equipment is sold by Comdel, Inc., Gloucester, Mass., USA. The power supply 126 can be attached to the COMDEL® matching network 125 via a coaxial cable 127. Two capacitance manometers (0-1 Torr and 0-100 Torr) (not shown) can be attached to the vacuum conduit 119 (also referred to as a pump line) to measure the process pressures.

The apparatus shown in FIG. 2 for treating the vessel 105 can be the same as that of FIG. 1, but as illustrated has more than one gas inlet conduit 111 to accommodate more than one vessel 105 in a single treatment cycle.

The apparatus shown in FIG. 1 or 2 optionally includes a vacuum bypass line 128 as shown in FIG. 3.

Optionally in any embodiment, lab ware configured as a flask, a bottle, or a tube can be processed in apparatus like that of FIGS. 1-3.

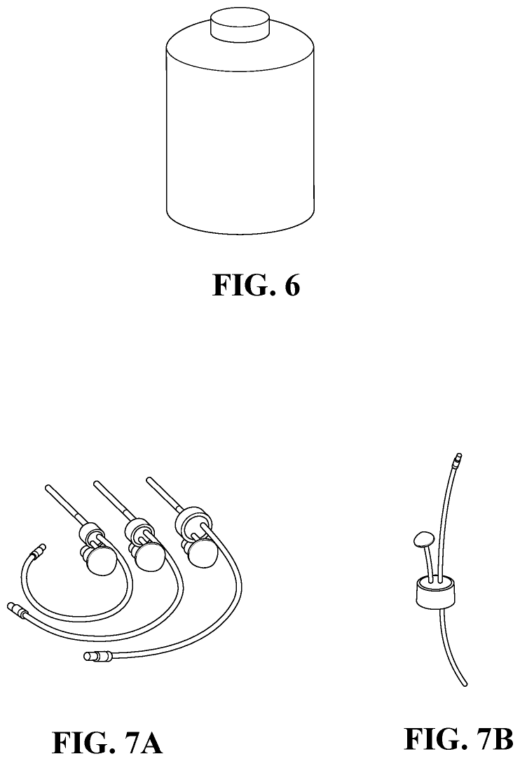

Optionally in any embodiment, lab ware configured as a plate, a microplate, a dish, or other object having relatively flat exterior surfaces to be treated can be treated in apparatus like that of FIGS. 1-3, but adapted to process flatter pieces. Optionally in any embodiment, the interior of the ceramic chamber 115 as illustrated here can be adapted as shown in FIG. 6 of WO 2016/176561 to support multiple microplates or other relatively flat objects during treatment as described in this specification. Optionally in any embodiment, the microplates or other flat objects can be oriented so the surface to be treated faces the center of the ceramic chamber 115, facilitating the application of plasma energized gas directly to the surfaces presented for treatment.

The process optionally improves cell recovery of a chicken embryo cell culture from the treated contact surface 102, relative to the initial contact surface 102, resulting in cell recovery from the treated contact surface 102 of at least 140% of the cells provided to the treated contact surface 102 at the beginning of the cell recovery test.

The cells can also grow on microcarrier surfaces, another type of substrate that also increases the contact surface area. A microcarrier is a support matrix allowing for adhesive cell growth. Microcarriers are usually 125-250 micrometer spheres (beads) and their density allows them to be maintained in suspension in the medium with gentle stirring. Microcarriers or beads can be made from a number of different materials including DEAE-dextran, glass, polystyrene plastic, acrylamide, collagen, and alginate. These microcarrier or bead materials, along with different surface chemistries, can influence cellular behavior, including morphology and proliferation. There are many advantages by using microbarriers (or beads) technologies, e.g. less culture medium and less lab ware needed.

While it is important to enhance cell adhesion and cell growth, it is equally important to harvest the cells and retain the quality of the cells after the completion of the growth process. Optionally, when microcarriers are used, cell harvesting can be considered to involve two steps: firstly, the cells are detached from microcarriers to produce a cell-microcarrier suspension; and secondly, a further separation step leaving the cells in suspension without the microcarriers present.

Typically, the first step, i.e. cell detachment from microcarriers is accomplished by enzymatic digestion. Different enzymes can be used based on the types of microcarriers, types of cells, etc. The enzymes can be, for example, trypsin, accutase, collagenase or a trypsin-accutase mixture. During the second step, filters or centrifuges are used to separate the cells from the microcarriers.

The present invention also optionally relates to, plasma coating or treatment of the microcarrier (e.g. bead) surface to provide high hydrophilic surface to enhance cell adhesion and cell growth. The coating or treatment does not have negative impact on the cell integrity during the cell adhesion, cell growth and cell recovery process.

### Cell Culture, Cell Harvest and Recovery Protocol

The following materials, equipment, and methods are contemplated for use with the present disclosure. Materials: CELLTREAT® 1,000 mL Roller Bottle (Product 229582), CELLTREAT® T-182 Flasks (Product 229351), Medium DMEM (Gibco; Ref #1995-065), Calf serum (Gibco; Ref@ 16170-078), 1×PBS (Gibco; Ref #14190-136), 1× Trypsin with 0.18 mM EDTA diluted with 1×PBS (Gibco; Ref #25200-056), Counting slides (Bio-Rad; Cat #145-0011), Cell counter (Bio-Rad; Model TC10), Trypan Blue Solution 0.4% (Armesco, Code:K940-100ML), Penicillin Streptomycin Soln, 100× (Corning; Ref #30-002C1), competitor 2,000 mL Roller Bottle.

The selected cells were counted and split into T-182 Flasks (3/33)×15 when received on Friday. On Monday, the 15× T-182 Flasks of cells were pooled. 10 mL of cells were added to the 1 L roller bottles and 20 mL of cells were added to the 2 L roller bottles. Roller bottles were rotated at 0.25 rpm in a humidified chamber at 39° C. with 5% CO2 in air. After 48 hours, the cells were harvested.

The harvesting of cells was performed in the following manner for 1 L roller bottles. The medium was decanted. The cells were rinsed with 25 mL of 1×PBS. Then 10 mL 1× Trypsin with 0.18 mM EDTA was added and incubated for 10 minutes. Finally 40 mL of complete medium was added. A 1 mL sample was collected and a cell count was performed.

For 2 L roller bottles, harvesting cells was performed as follows. The medium was decanted. The cells was rinsed with 50 mL of 1×PBS. Then 20 mL 1× Trypsin with 0.18 mM EDTA was added and incubated for 10 minutes. 80 mL of complete medium was added. 1 mL sample was collected and a cell count was performed.

Each sample was diluted 10× to help separate the cells. The cell samples were once again diluted 10× but in addition with 0.4% Trypan Blue to a 1:1 ratio. The 10 μL of the cell/trypan blue sample was loaded into a counting slide, which was loaded into the Bio-Rad Cell Counter and recorded.

Analysis performed compared Viable Cell Recovery, which is calculated as follows:

% Viable Cell Recovery=Total Viable Cells Harvested/Initial Total Viable Cells

Materials List

| Material | Manufacturer/Cat# |
|---|---|
| Medium DMEM | Gibco; Ref# 11995-065 |
| Calf serum | Gibco; Ref# 16170-078 |
| 1x PBS | Gibco; Ref# 14190-136 |

1x Trypsin w/0.18 mM EDTA diluted with 1x PBS from: Gibso; Ref#
25200-056

| | |
|---|---|
| Counting slides | Bio-Rad; Cat# 145-0011 |
| Cell counter | Bio-Rad; Model TC10 |
| Trypan Blue solution 0.4% | Amresco; Code: K940-100 ML |
| Penicillin Streptomycin Soln, 100X | Coming; Ref# 30-002-C1 |
| T-182 flask | Celltreat; Code: 229351 |

Experiment Design
Order cells so that they arrive on Friday. Once cells
are received, count and split to 1-182 (3/33) x15.
Monday pool 15 t-182 flasks and add 10 ml of
cells/1 L roller bottles and 20 ml/2 L roller bottles.
Roller bottles are rotated at 0.25 rpm in a humidified
chamber at 39° C. with 5% CO2 in air.
Wednesday the cells are harvested (total 48 hours).
In order to harvest:

| | 1 L Roller bottles | 2 L Roller bottles |
|---|---|---|
| 1 | Decant medium | Decant medium |
| 2 | Rinse with 25 ml 1x PBS | Rinse with 50 ml 1x PBS |
| 3 | Add 10 ml 1x | Add 20 ml 1x |
| | Trypsin with 0.18 mM EDTA | Trypsin w/0.18 mM EDTA |
| 4 | Incubate for 10 minutes | Incubate for 10 minutes |
| 5 | Add 40 ml of Complete medium | Add 80 ml of Complete medium |
| 6 | Collect 1 ml sample and count | Collect 1 ml sample and count |

Each sample is mixed 10x to help separate the cells. The cell samples are once again mixed 10x but this time with 0.4% Trypan Blue to a 1:1 ratio. 10 μl of the cell/trypan blue sample is then loaded into accounting slide and loaded into the BioRad Cell Counter and recorded.

Example 1

This experiment was carried out to examine the cell recovery (i.e. cell growth) improvement and contact angles due to the present surface treatment applied to a 1 L CellTreat roller bottle made of polystyrene. This experiment also compared the treatment of the current invention with competitive treatments, such as the Corning Tissue Culture Treated (TCT) roller bottle and Corning Cellbind roller bottle, regarding cell growth. The cell line for the test was chicken embryo cells. The treatment process is described in the specification. Roller bottles 1-4 were treated according to the current invention and the parameters used are shown in Table 1a. The treated bottles were then loaded with cells as shown in Table 1b The results in Tables 2-4 show that the treatment of roller bottle 2, sometimes referred to in this specification as treatment 2, consistently afforded the best cell growth results (expressed in cell recovery data). The surface analysis shown in the following examples was performed on the roller bottles treated with the method of treatment 2, unless specified otherwise.

Water contact angles were also determined, as reported in Table 5.

TABLE 1a
Treatment Parameters

| Roller Bottle | Nitrogen | Oxygen | Power (W) | Time (s) |
|---|---|---|---|---|
| 1 | 10 | 20 | 475 | 60 |
| 2 | 10 | 10 | 600 | 60 |
| 3 | 0 | 20 | 400 | 60 |
| 4 | 0 | 10 | 500 | 90 |

TABLE 1b
Starting Cell Loading

| | Starting Viable | Starting Cells | Starting Viable |
|---|---|---|---|
| Cell Loading | (×10⁵) | (×10⁵) | (%) |
| 1^{st} round | 96.0 | 105.0 | 92.0 |
| 2nd round | 24.9 | 35.4 | 70.0 |
| 3rd round | 95.0 | 104.0 | 91.0 |

TABLE 2
Cell Recovery Results (1^{st} Round)

| Roller Bottle | Viability % | Recovery % |
|---|---|---|
| 1 | 90% | 143% |
| 2 | 81% | 228% |
| Corning TCT | 82% | 130% |
| (2 Liter) | | |
| Corning CellBIND | 87% | 113% |
| (2 Liter) | | |

TABLE 3
Cell Recovery Results (2^{nd} Round)

| Roller Bottle | Viability % | Recovery % |
|---|---|---|
| 1 | 96% | 162% |
| 2 | 79% | 190% |
| Corning TCT | 93% | 149% |
| (2 Liter) | | |
| Corning CellBIND | — | — |
| (2 Liter) | | |

TABLE 4
Cell Recovery Results (3^{rd} Round)

| Roller Bottle | Viability % | Recovery % |
|---|---|---|
| 2 | 91% | 81% |
| 3 | 76% | 76% |
| 4 | 82% | 63% |
| Corning TCT | 91% | 64% |
| (2 Liter) | | |
| Corning CellBIND | 94% | 63% |
| (2 Liter) | | |

Table 5: Water Contact Angles

TABLE 5
Water Contact Angles

| Roller Bottle | Surface Contact Angle |
|---|---|
| 1 | 61° |
| 2 | 52° |
| 3 | 39° |
| 4 | 38° |
| Corning TC (2 Liter) | 62° |
| Corning CellBIND (2 Liter) | 39° |

### Example 2. XPS Surface Analysis of Treated Roller Bottle of the Invention and Untreated Roller Bottle

This example was carried out to determine the chemical composition and chemical bonding of the contact surface of an untreated 1 L CELLTREAT™ roller bottlemade of polystyrene and the contact surface of a treated otherwise identical roller bottle B. The surface treating process for the roller bottle of FIG. 6 is the same as treatment 2 in Example 1. The XPS was performed on one area (the middle area) of the contact surface of bottle A and four areas of the contact surface of bottle B. The four areas are shown in FIG. 6. The concentration of the elements was determined from high resolution spectra. These XPS results are summarized in Table 6.

TABLE 6
Atomic Concentrations (in atomic %)

| Sample | C | N | O | Si |
|---|---|---|---|---|
| Bottle A (middle) | 90.1 | 0.1 | 7.4 | 2.4 |
| Bottle B (top) | 71.1 | 0.6 | 22.8 | 5.4 |
| Bottle B (middle) | 72.4 | 0.7 | 21.9 | 5.0 |
| Bottle B (bottom) | 73.6 | 0.6 | 21.2 | 4.6 |
| Bottle B (base) | 71.2 | 0.6 | 22.7 | 5.5 |

The results show that the treatment of the current invention results in three times more oxygen on the treated surface than on an identical untreated surface.

The chemical bonding information is shown in Table 7.

TABLE 7
Carbon and Silicon Chemical States (in atomic %)

| | Carbon | | | | | | | |
|---|---|---|---|---|---|---|---|---|
| | | | | | | aromatic | Silicon | |
| Sample | C—(C, H) | C—O | C═O | O—C═O | CO₃ | loss | silicone | silicate |
| Bottle A (middle) | 80.0 | 4.9 | 1.0 | — | — | 4.1 | 1.9 | 0.6 |
| Bottle B (top) | 59.3 | 6.8 | 2.2 | 0.7 | 1.2 | 1.0 | 3.2 | 2.1 |
| Bottle B (middle) | 61.7 | 7.0 | 2.0 | 0.4 | 0.8 | 0.5 | 3.0 | 2.0 |
| Bottle B (bottom) | 61.8 | 7.2 | 2.4 | 0.8 | 0.8 | 0.6 | 2.8 | 1.8 |
| Bottle B (base) | 59.4 | 6.7 | 2.3 | 1.0 | 0.9 | 1.0 | 3.2 | 2.3 |

The chemical states of the detected elements were determined from the high resolution spectra. For the elements C and Si, the spectra were curve fit to estimate the relative amounts of each element in different oxidation states. The curve fit results are shown on the individual spectra and summarized in Table 7.

### Example 3. XPS in-Depth Analysis of Treated Roller Bottle of the Invention and Untreated Roller Bottle

This example was used to determine the in-depth chemical composition of the contact surface of the treated roller bottle B of this invention.

Survey spectra were acquired for the contact surface of the treated roller bottle B. A depth profile was acquired using 1 kV Ar+. The results are shown in Table 8. This beam voltage was selected to minimize preferential sputtering of oxygen atoms. While this minimizes preferential sputtering, it does not completely remove this artifact. Consequently, the oxygen concentrations during the depth profiles are expected to be higher than the measured values. Note that the depth scales in this study assumed that the samples sputtered at the same rate as a 825A spin cast thin film of polystyrene.

TABLE 8
Atomic Concentration

| Depth (Å) | C | N | O | Si |
|---|---|---|---|---|
| 0.0 | 72.5 | 0.4 | 22.4 | 4.7 |
| 5.8 | 94.0 | — | 3.9 | 2.1 |
| 11.5 | 97.5 | — | 1.4 | 1.1 |
| 17.3 | 98.5 | — | 0.8 | 0.7 |
| 23.0 | 98.7 | — | 0.6 | 0.7 |
| 28.8 | 98.9 | — | 0.6 | 0.5 |
| 34.6 | 99.3 | — | 0.3 | 0.4 |
| 40.3 | 99.4 | — | 0.3 | 0.3 |
| 46.1 | 99.3 | — | 0.3 | 0.4 |
| 51.8 | 99.5 | — | 0.3 | 0.2 |
| 57.6 | 99.5 | — | 0.3 | 0.2 |
| 63.4 | 99.6 | — | 0.3 | 0.2 |
| 69.1 | 99.5 | — | 0.4 | 0.2 |
| 74.9 | 99.5 | — | 0.4 | 0.1 |
| 80.6 | 99.5 | — | 0.4 | — |
| 86.4 | 99.5 | — | 0.5 | — |
| 95.0 | 99.7 | — | 0.3 | — |
| 104 | 99.6 | — | 0.4 | — |
| 112 | 99.7 | — | 0.3 | — |
| 121 | 99.6 | — | 0.4 | — |
| 130 | 99.7 | — | 0.3 | — |
| 143 | 99.6 | — | 0.4 | — |
| 156 | 99.8 | — | 0.2 | — |
| 168 | 99.5 | — | 0.5 | — |
| 181 | 99.7 | — | 0.3 | — |
| 194 | 99.8 | — | 0.2 | — |
| 214 | 99.8 | — | 0.2 | — |

^{a} Normalized to 100% of the elements detected. XPS does not detect H or He.
^{b} A dash line indicates the element is not detected.

While the technology has been described in detail and with reference to specific examples and embodiments thereof, it will be apparent to one skilled in the art that various changes and modifications can be made therein without departing from the spirit and scope thereof. Additional disclosure is provided in the claims, which are considered to be a part of the present description, each claim defining an optional and optional embodiment.

1. A method comprising:
providing a polymeric substrate including an initial contact surface and an interior portion adjacent to the initial contact surface,
contacting the initial contact surface with a process gas; and
introducing radio frequency electrical power in the process gas adjacent to the initial contact surface to generate plasma adjacent to the initial contact surface, thereby forming a treated polymeric substrate having a treated contact surface, under conditions effective to improve cell recovery of a chicken embryo cell culture from the treated contact surface, relative to the initial contact surface, resulting in cell recovery from the treated contact surface of at least 140% of the cells provided to the treated contact surface at the beginning of the cell recovery test.

2. The method of claim 1, in which the x-ray photoelectron spectroscopy (XPS) atomic composition of the treated contact surface is:
from 10% to 25% oxygen, from 0 to 5% nitrogen, and from 70% to 90% carbon;
optionally from 15% to 24% oxygen, from 0.1% to 5% nitrogen, and from 70% to 80% carbon;
optionally from 20% to 24% oxygen, from 0.1% to 1% nitrogen, and from 70% to 79% carbon.

3. The method of claim 1, in which the XPS atomic composition of the interior portion of the treated polymeric substrate comprises less oxygen and more carbon than the treated contact surface.

4. The method of claim 1, in which the XPS atomic composition of the interior portion of the treated polymeric substrate at a depth of 0.6 nm comprises from 1% to 10% oxygen.

5. The method of claim 1, in which the XPS atomic composition of the interior portion of the treated polymeric substrate at a depth of 1.2 nm comprises from 0.5% to 5% oxygen.

6. The method of claim 1, in which the XPS atomic composition of the interior portion of the treated polymeric substrate at a depth of 1.7 nm comprises from 0.3% to 3% oxygen.

7. The method of claim 1, in which the XPS atomic composition of the interior portion of the treated polymeric substrate at a depth of 2.3 nm comprises from 0.1% to 1% oxygen.

8. The method of claim 1, in which the XPS atomic composition of the interior portion of the treated polymeric substrate at a depth of 2.9 nm comprises from 0.1% to 1% oxygen.

9. The method of claim 1, in which the viability of a chicken embryo cell culture grown in contact with the treated contact surface and harvested, relative to the initial contact surface, is at least 88%, optionally from 88% to 99%, optionally from 88% to 97%, optionally from 94% to 96%.

10. The method of claim 1, in which the recovery of a chicken embryo cell culture grown in contact with the treated contact surface and harvested, relative to the initial contact surface, is at least 132%, optionally from 132% to 300%, optionally from 140% to 250%, optionally from 140% to 230%.

11. The method of claim 1, in which the surface contact angle of the treated contact surface is from 38° to 62°, optionally from 50° to 70°, optionally from 55° to 65°, optionally from 60° to 64°, optionally from 30° to 50°, optionally from 30 to 40°, optionally from 35° to 45°, optionally from 37° to 41°.

12. The method of claim 1, in which the treated polymeric substrate comprises a vessel having a wall having an inner surface enclosing a lumen, an outer surface, and an interior portion between and spaced from at least the inner surface and the outer surface.

13. The method of claim 12, in which the inner surface is generally cylindrical.

14. The method of claim 12, in which the treated contact surface comprises at least a portion of the inner surface of the vessel.

15. The method of claim 12, in which the vessel comprises a roller bottle.

16. The method of claim 15, in which the roller bottle comprises an inner surface defining the treated contact surface, the inner surface having multiple ribs.

17. The method of claim 12, in which the vessel has a volumetric capacity from 1 mL to 100 L, optionally from 100 mL to 5 L, optionally about 1 L, optionally about 2 L.

18. The method of claim 1, in which the treated polymeric substrate comprises a plate, a dish, a flask, a bottle, or a tube.

19. The method of claim 1, in which the treated polymeric substrate comprises thermoplastic material, for example a thermoplastic resin, for example an injection-molded thermoplastic resin.

20. The method of claim 19, in which the thermoplastic material comprises a hydrocarbon polymer, for example an olefin polymer, polypropylene (PP), polyethylene (PE), cyclic olefin copolymer (COC), cyclic olefin polymer (COP), polymethylpentene, polystyrene, hydrogenated polystyrene, polycyclohexylethylene (PCHE), or combinations of two or more of these, or a heteroatom-substituted hydrocarbon polymer, for example a polyester, polyethylene terephthalate (PET), polyethylene naphthalate, polybutylene terephthalate (PBT), polyvinylidene chloride (PVdC), polyvinyl chloride (PVC), polycarbonate, polylactic acid, epoxy resin, nylon, polyurethane polyacrylonitrile, polyacrylonitrile (PAN), an ionomeric resin, or any combination, composite, blend, or laminate of any two or more of the above materials.

21. The method of claim 20, in which the thermoplastic resin comprises polystyrene.

22. The method of claim 1, in which the process gas comprises oxygen atoms, nitrogen atoms, or both oxygen and nitrogen atoms, and preferably comprises oxygen, nitrogen, nitrous oxide, or a combination of any two or more of these.

23. The method of claim 1, in which the process gas is essentially free of water.

24. The method of claim 1, in which the surface is contacted with a process gas by conveying the process gas through a gas inlet conduit having an outlet adjacent to the initial contact surface.

25. The method of claim 1, in which the radio frequency is from 1 to 50 MHz, optionally 13.56 MHz.

26. The method of claim 1, in which the radio frequency electrical power used to excite the plasma is from 1 to 1000 Watts, optionally from 100 to 900 Watts, optionally from 50 to 600 Watts, optionally 200 to 700 Watts, optionally 400 to 600 Watts, optionally 100 to 500 Watts, optionally from 500 to 700 Watts, optionally from 1 to 100 Watts, optionally from 1 to 30 Watts, optionally from 1 to 10 Watts, optionally from 1 to 5 Watts.

27. The method of claim 1, in which the radio frequency electrical power is introduced at least in part by an external applicator generally surrounding the initial contact surface.

28. The method of claim 1, in which the treated polymeric substrate is a vessel comprising an inner surface defining a lumen, and the radio frequency electrical power is introduced at least in part by an internal applicator located at least partially within the lumen.

29. The method of claim 28, in which the internal applicator located at least partially within the lumen further comprises a gas inlet conduit for contacting the initial contact surface with the process gas.
